(19) United States
(12) Patent Application Publication
(10) Pub. No.: US 2021/0207071 A1
(43) Pub. Date: Jul. 8, 2021

(54) CELL CULTURING DEVICE AND CELL CULTURING METHOD

(71) Applicant: OLYMPUS CORPORATION, Tokyo (JP)

(72) Inventors: Koh MOHRI, Tokyo (JP); Shintaro TAKAHASHI, Tokyo (JP); Asuka NAKAMURA, Tokyo (JP); Shoichi KANEKO, Tokyo (JP); Yoshihito IGUCHI, Tokyo (JP); Shogo USUI, Tokyo (JP); Takayuki NAKATOMI, Tokyo (JP)

(73) Assignee: OLYMPUS CORPORATION, Tokyo (JP)

(21) Appl. No.: 17/209,846

(22) Filed: Mar. 23, 2021

Related U.S. Application Data
(63) Continuation of application No. PCT/JP2018/036802, filed on Oct. 2, 2018.

(51) Int. Cl. C12M 1/34; C12M 1/36

(52) U.S. Cl. CPC C12M 41/26; C12M 41/48

(57) ABSTRACT

A cell culturing device includes: a sensor that measures a pH of a medium in which cells are being cultured; and one or more processors including hardware. The one or more processors are configured to: measure an elapsed time since the cells were seeded in the medium; determine whether the measured pH is lower than a prescribed threshold and whether the measured elapsed time has passed a prescribed time; and issue an instruction for medium replacement in response to determining that the pH is lower than the prescribed threshold or in response to determining that the elapsed time has passed the prescribed time.

### CROSS-REFERENCE TO RELATED APPLICATIONS

This is a continuation of International Application PCT/JP2018/036802, with an international filing date of Oct. 2, 2018, which is hereby incorporated by reference herein in its entirety.

### TECHNICAL FIELD

The present invention relates to a cell culturing device and a cell culturing method.

### BACKGROUND ART

In the related art, there are known cell culturing devices and cell culturing methods for culturing cells (for example, see Patent Literature 1). The cell culturing device described in Patent Literature 1 includes a measuring portion that measures the pH of a medium in a culturing vessel and an instructing portion that issues an instruction for replacing the medium in the culturing vessel.

Also, with the cell culturing device and the cell culturing method described in Patent Literature 1, because there are cases in which cell culturing becomes inactive when the pH of a medium decreases and because the amount of time until the pH of the medium reaches a certain level normally decreases with an increase in the number of cells, the medium is efficiently replaced in response to the instructing portion issuing, at the time of a first medium replacement since the cell seeding, a replacement instruction on the basis of the pH of the medium measured by the measuring portion and in response to the instructing portion issuing, in second and subsequent medium replacements, the replacement instruction on the basis of the pH of the medium measured by the measuring portion and a time interval with respect to the previous medium replacement.

### CITATION LIST

### Patent Literature

{PTL 1} PCT International Publication No. WO 2015/098080

### SUMMARY OF INVENTION

An aspect of the present invention is directed to a cell culturing device including: a sensor that measures a pH of a medium in which cells are being cultured; and one or more processors comprising hardware, the one or more processors being configured to: measure an elapsed time since the cells were seeded in the medium; determine whether the measured pH is lower than a prescribed threshold and whether the measured elapsed time has passed a prescribed time; and issue an instruction for medium replacement in response to determining that the pH is lower than the prescribed threshold or in response to determining that the elapsed time has passed the prescribed time.

Another aspect of the present invention is directed to a cell culturing method including: seeding cells in a medium; measuring a pH of the seeded medium; measuring an elapsed time since the cells were seeded in the medium; determining whether the measured pH is lower than a prescribed threshold and whether the measured elapsed time has passed a prescribed time; and replacing the medium in response to determining that the pH is lower than the prescribed threshold or in response to determining that the elapsed time has passed the prescribed time.

Another aspect of the present invention is directed to a non-transitory computer readable medium storing a cell culturing program for causing a computer to execute: receiving a pH of a medium in which cells are being cultured; measure an elapsed time since the cells were seeded in the medium; determining whether the received pH is lower than a prescribed threshold and whether the measured elapsed time has passed a prescribed time; and issuing an instruction for medium replacement in response to determining that the pH is lower than the prescribed threshold or in response to determining that the elapsed time has passed the prescribed time.

### BRIEF DESCRIPTION OF DRAWINGS

FIG. 1 is a schematic configuration diagram of a cell culturing device according to a first embodiment of the present invention.

FIG. 2 is a block diagram of the cell culturing device in FIG. 1.

FIG. 3 is a longitudinal sectional view of a culturing vessel and a pH measuring portion in FIG. 1.

FIG. 4 is a diagram showing an example of a replacement-determining operation mode.

FIG. 5 is an example of a time-series curve of the pH of a medium recorded by a recording portion.

FIG. 6 is a flowchart for explaining a cell culturing method according to the first embodiment of the present invention.

FIG. 7 is a schematic configuration diagram of a cell culturing device according to a second embodiment of the present invention.

FIG. 8 is a block diagram of the cell culturing device in FIG. 7.

FIG. 9 is a schematic configuration diagram of a cell culturing device according to a modification of the second embodiment of the present invention.

FIG. 10 is a schematic diagram showing a configuration of a multiwell plate and a pH measuring portion in FIG. 9.

FIG. 11 is a schematic configuration diagram of a cell culturing device according to another modification of the second embodiment of the present invention.

FIG. 12 is a schematic diagram showing a configuration of a multiwell plate and a pH measuring portion in FIG. 11.

### DESCRIPTION OF EMBODIMENTS

A cell culturing device and a cell culturing method according to a first embodiment of the present invention will be described below with reference to the drawings.

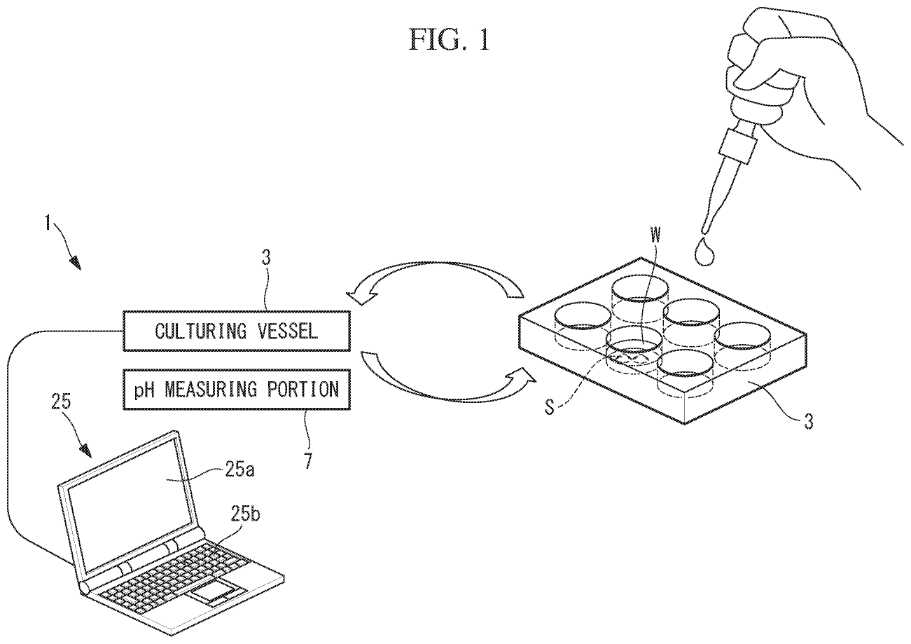

As shown in FIG. 1, a cell culturing device 1 according to this embodiment is, for example, a device with which, in response to issuing of an instruction for replacing a medium W in a culturing vessel 3 in which cells S are being cultured, a user manually replaces the medium W on the basis of the instruction.

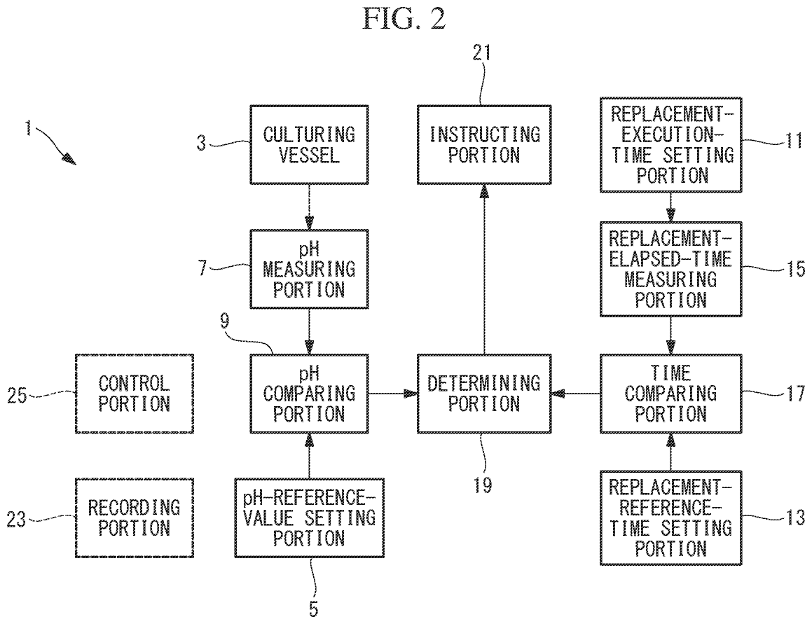

As shown in FIG. 2, the cell culturing device 1 includes, for example, a pH-reference-value setting portion 5, a pH measuring portion 7, a pH comparing portion (determining portion) 9, a replacement-execution-time setting portion 11, a replacement-reference-time setting portion (setting portion) 13, a replacement-elapsed-time measuring portion (time measuring portion) 15, a time comparing portion (determining portion) 17, a determining portion (determining portion) 19, an instructing portion (replacement instructing portion) 21, a recording portion 23, and a control portion (storage portion, input portion, notifying portion) 25.

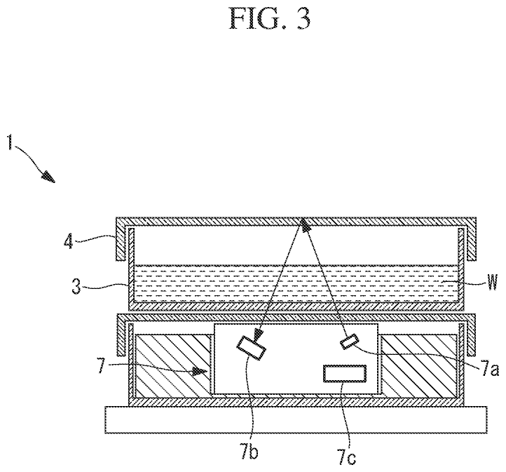

As shown in FIG. 3, the culturing vessel 3 is, for example, a bottomed cylindrical well vessel or dish vessel that is open at the top and is capable of retaining the medium W in the interior thereof. The opening at the top portion of the culturing vessel 3 is closed with a top lid 4. In the state in which the medium W is retained in the culturing vessel 3 and in which the cells S are seeded in the medium W, the cells S are cultured by maintaining the temperature, the concentration, and so forth of the medium W in prescribed culturing conditions. The medium W is, for example, a culturing liquid that contains a large quantity of a nutrient substance.

The control portion 25 controls the operations of the above-described respective constituent elements of the cell culturing device 1. The control portion 25 is, for example, a computer including: a CPU (central processing unit) that controls the overall operation of the cell culturing device 1 by executing a control program; a main memory that is used by the CPU as a working memory, as needed; an instruction acquisition portion that acquires various types of instructions from a user; an interface unit that manages the transfer of various types of data among the above-described respective constituent elements of the cell culturing device 1; and an auxiliary storage device, such as a hard disk drive that stores various types of programs and data (all of which are not shown). In addition, the control portion 25 may include: a pH-reference-value acquisition portion that acquires a pH reference value from the pH-reference-value setting portion 5; a pH acquisition portion that acquires a pH value of the medium from the pH measuring portion 7; the pH comparing portion (determining portion) 9; a replacement-time acquisition portion that acquires a replacement execution time from the replacement-execution-time setting portion 11; a replacement-reference-time acquisition portion that acquires a replacement reference time from the replacement-reference-time setting portion (setting portion) 13; the replacement-elapsed-time measuring portion (time measuring portion) 15; the time comparing portion (determining portion) 17; the determining portion (determining portion) 19; and the instructing portion (replacement instructing portion) 21.

Processing in each of the above-described constituent elements is realized as a result of the CPU of the control portion 25 reading out prescribed application programs stored in the auxiliary storage device to the main memory and executing said programs. In addition, the CPU of the control portion 25 determines whether or not the number of days for which the cells S have been cultured has reached a set number of culturing days set in advance.

In addition, as shown in FIG. 1, the control portion 25 includes: a display portion 25a such as a display; and an input portion 25b, such as a mouse or a keyboard, that allows a user to input information such as a threshold for replacing the medium and the timing at which the medium was replaced.

In addition, the control portion 25 has a function for externally issuing an instruction for replacing the medium. For example, the control portion 25 displays a medium-replacement instruction message on the display portion 25a, issues a medium-replacement instruction alarm, or forwards an e-mail with the medium-replacement instruction.

The pH-reference-value setting portion 5 sets the pH reference value (prescribed threshold) that serves as the reference for replacing the medium W. Hereinafter, the pH reference value for the medium W will be referred to as the pH replacement reference value. The pH replacement reference value is arbitrarily determined by the user and is input by means of the input portion 25b. The pH replacement reference value is, for example, 6.9.

The pH measuring portion 7 is a device that measures the pH of the medium W in which the cells S are being cultured and is disposed, for example, below the culturing vessel 3, as shown in FIG. 3. In addition, the pH measuring portion 7 also optically measures the color of the medium W and includes: a light source 7a that emits light; an optical sensor 7b detects light; and a computing portion 7c that calculates the pH of the medium W.

The pH measuring portion 7 measures the light transmittances at the medium W by means of the optical sensor 7b by irradiating the medium W, from therebelow, with light beams having different wavelengths by means of the light source 7a and by detecting, by means of the optical sensor 7b, transmitted light that has been reflected by the top lid 4 of the culturing vessel 3 after passing through the medium W. Also, by means of the computing portion 7c, the pH of the medium W is calculated on the basis of differences among the transmittances detected by the optical sensor 7b for the respective wavelengths.

The pH comparing portion 9 compares the pH of the medium W measured by the pH measuring portion 7 with the pH replacement reference value set by the pH-reference-value setting portion 5.

When the user inputs, by means of the input portion 25b, the time at which the medium was replaced, the replacement-execution-time setting portion 11 records said time, thus setting the most-recent medium replacement time, that is, the time at which the medium was replaced in the immediate past.

The replacement-reference-time setting portion 13 sets a first reference time and a second reference time (both of which are prescribed times) for the replacement times that serve as the references for replacing the medium W and inputs the set times to the determining portion 19. Hereinafter, the first reference time for the replacement time of the medium W will be referred to as the first replacement reference time, the second reference time will be referred to as the second replacement reference time, and the two reference times as a whole will simply be referred to as the replacement reference time.

The first replacement reference time is the prescribed time at which the medium is replaced for the first time after the cells S are seeded in the medium W. The first replacement reference time is arbitrarily determined by the user on the basis of the time that is recommended for each of prescribed first culturing conditions and is input by means of the input portion 25b. The first culturing conditions are, for example, the type of cells S to be cultured, the type of culturing vessel 3 in which the cells S are accommodated, the type of coating agent that coats the culturing vessel 3, the type of culturing procedure, and so forth, as well as the type of the medium W to be used.

The second replacement reference time is the prescribed time from the medium replacement in the immediate past to the next medium replacement. The second replacement reference time is arbitrarily determined by the user, for example, on the basis of the time that is recommended for each of prescribed second culturing conditions and is input by means of the input portion 25b. The second culturing conditions are, for example, the type of medium W to be used. Hereinafter, the first culturing conditions and the second culturing conditions as a whole will simply be referred to as the culturing conditions.

The replacement-elapsed-time measuring portion 15 measures the elapsed time since the cells S were seeded in the medium W. In addition, the replacement-elapsed-time measuring portion 15 also measures, on the basis of the most-recent medium replacement time set by the replacement-execution-time setting portion 11, the elapsed time since the most-recent medium replacement each time the medium is replaced.

The time comparing portion 17 compares the elapsed time measured by the replacement-elapsed-time measuring portion 15 with the first replacement reference time and the second replacement reference time set by the replacement-reference-time setting portion 13.

The determining portion 19 determines the pH of the medium W and determines the elapsed time. Specifically, the determining portion 19 reads out one of the comparison result from the pH comparing portion 9 and the comparison result from the time comparing portion 17 in accordance with prescribed replacement-determining operation modes and determines, on the basis of the read-out comparison result, whether or not the pH of the medium W is lower than the pH replacement reference value or whether or not the elapsed time has passed the first replacement reference time or the second replacement reference time. The replacement-determining operation mode is selected by the user by means of the input portion 25b. The determination result from the determining portion 19 is transmitted to the instructing portion 21.

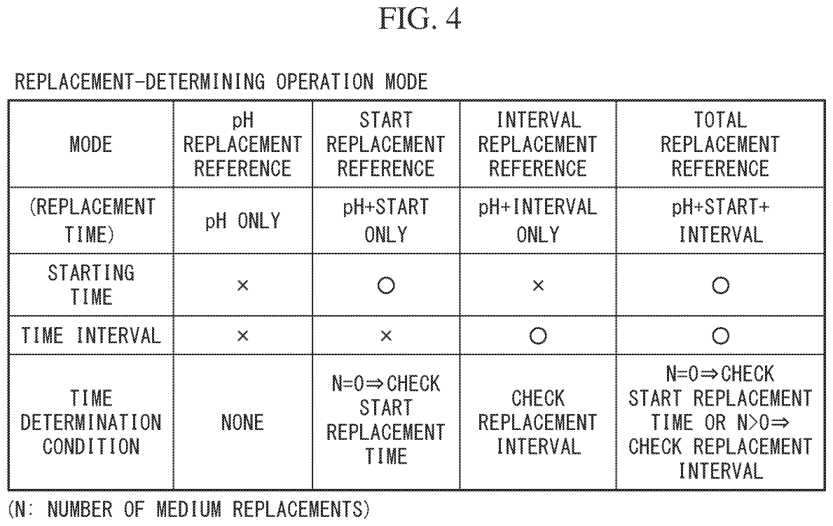

As shown in FIG. 4, examples of the replacement-determining operation modes include: a pH replacement reference mode for determining the timing at which the medium is replaced with reference to only the pH of the medium W; a start replacement reference mode for determining the timing at which the medium is replaced with reference to only the pH of the medium W and a first prescribed time since the culturing is started; an interval replacement reference mode for determining the timing at which the medium is replaced with reference to only the pH of the medium W and a prescribed time interval of the medium replacement; and a total replacement reference mode for determining the timing at which the medium is replaced with reference to all of the pH of the medium W, the first prescribed time since the culturing is started, and the prescribed time interval of the medium replacement.

The instructing portion 21 transmits a medium-replacement instruction to the control portion 25 in one of the case in which the determining portion 19 determines that the pH of the medium W is lower than the pH replacement reference value and the case in which the determining portion 19 determines that the elapsed time has passed the replacement reference time.

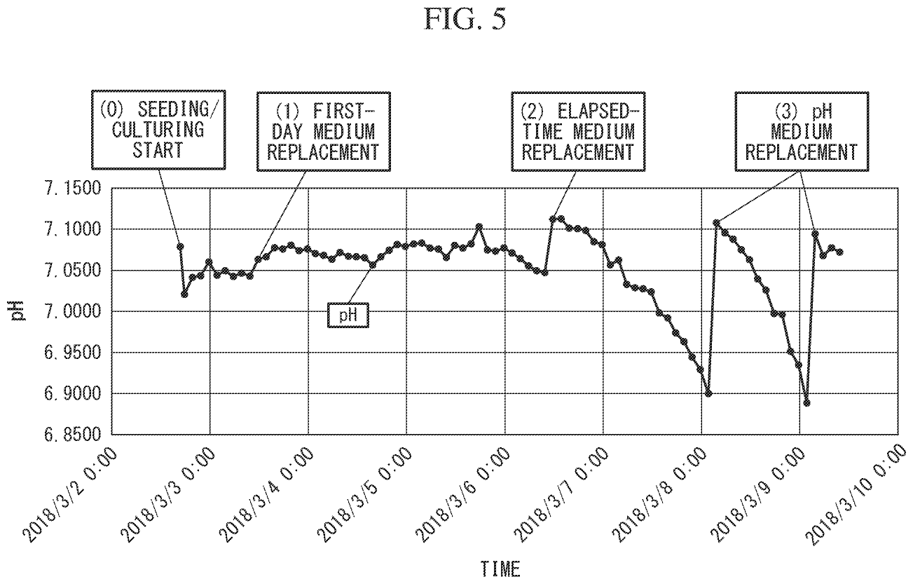

The recording portion 23 records, for example, as shown in FIG. 5, the pH of the medium W measured by the pH measuring portion 7 in a time series.

Next, with the cell culturing method according to this embodiment, cells S are seeded in the medium W, the pH of the medium W in which the cells S have been seeded is measured, the elapsed time since the cells S were seeded in the medium W is measured, it is determined whether or not the measured pH of the medium W is lower than the pH replacement reference value and whether or not the measured elapsed time has passed the first replacement reference time or the second replacement reference time, and the medium is replaced in one of the case in which the pH of the medium W is determined to be lower than the pH replacement reference value and the case in which the elapsed time is determined to have passed the first replacement reference time or the second replacement reference time.

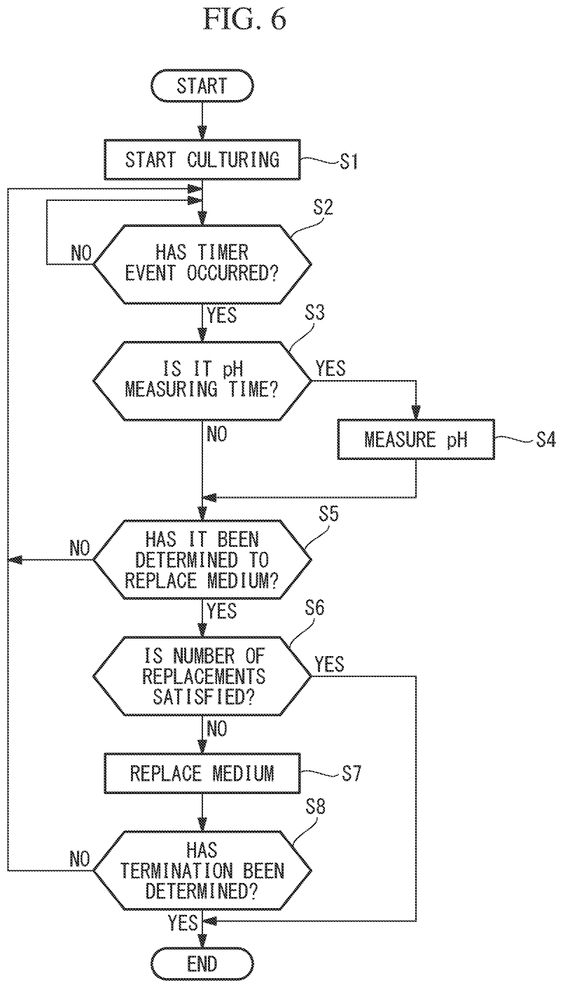

Next, the operation of the cell culturing device 1 and the cell culturing method according to this embodiment will be described with reference to the flowchart in FIG. 6.

In order to culture the cells S by means of the cell culturing device 1, having the above-described configuration, and the cell culturing method, first, the user selects one of the replacement-determining operation modes by means of the input portion 25b. In addition, the user inputs, by means of the input portion 25b, the pH replacement reference value and inputs the replacement reference time in accordance with the selected replacement-determining operation mode. In addition, the user sets the number of culturing days or culturing time.

Next, the cells S are seeded in the medium W in the culturing vessel 3, and the culturing of the cells S is started (step S1). In step S1, information such as the pH of the medium W and the elapsed time is reset and the number (N) of times the medium has been replaced is also reset. Then, the replacement-elapsed-time measuring portion 15 counts the elapsed time from the current time.

Next, events occur at a certain time interval, for example, every one minute (step S2), and the control portion 25 checks whether or not a target time has been reached. In the case in which it is time for measuring the pH of the medium W (“YES” in step S3), the pH measuring portion 7 measures the pH of the medium W in the culturing vessel 3 (step S4). In the case in which it is not time for measuring the pH (“NO” in step S3), the procedure directly proceeds to step S5.

The time comparing portion 17 compares the elapsed time measured by the replacement-elapsed-time measuring portion 15 and the replacement reference time set by the replacement-reference-time setting portion 13. In addition, in the case in which the pH of the medium W is measured by the pH measuring portion 7, the pH comparing portion 9 compares the measured pH of the medium W and the pH replacement reference value set by the pH-reference-value setting portion 5.

Next, the determining portion 19 determines, in accordance with the replacement-determining operation mode, whether or not the pH of the medium W is lower than the pH replacement reference value or whether or not the elapsed time since the cells S were seeded has passed the replacement reference time (step S5). In the case in which the determining portion 19 determines that the pH of the medium W is lower than the pH replacement reference value or in the case in which the determining portion 19 determines that the elapsed time since the cells S are seeded has passed the replacement reference time (“YES” in step S5), the control portion 25 determines whether or not a prescribed number of replacements has been satisfied (step S6).

The prescribed number of replacements is, for example, a set number of replacements+1 or the set number of replacements. As a result of setting the prescribed number of replacements to the set number of replacements+1, it is possible to end the culturing after performing sufficient culturing in a new medium W instead of ending the culturing immediately after replacement with the new medium W.

In the case in which the control portion 25 determines that the number of medium replacements has satisfied the prescribed number of replacements (“YES” in step S6), the culturing of the cells S is terminated. On the other hand, in the case in which the control portion 25 determines that the number of medium replacements has not satisfied the prescribed number of replacements (“NO” in step S6), the instructing portion 21 issues the instruction for medium replacement. The medium replacement instruction issued by the instructing portion 21 is displayed on, for example, the display portion 25a of the control portion 25. Accordingly, the user manually replaces the medium on the basis of the medium replacement instruction (step S7).

Next, the control portion 25 determines whether or not the number of culturing days has reached a set number of culturing days set in advance, that is, whether or not the culturing has been completed (step S8). In the case in which the control portion 25 determines that the number of culturing days has reached the set number of culturing days (“YES” in step S8), the culturing of the cells S is terminated. On the other hand, in the case in which the control portion 25 determines that the number of culturing days has not reached the set number of culturing days (“NO” in step S8), the procedure returns to step S2.

Here, the determination operation will be described for each of the replacement-determining operation modes.

In the case in which the pH replacement reference mode is selected, in step S5, the determining portion 19 reads out the comparison result of the pH of the medium W from the pH comparing portion 9, and, when it is determined that the pH of the medium W is lower than the pH replacement reference value, the instructing portion 21 issues the instruction for medium replacement.

In the case in which the start replacement reference mode is selected, in step S5, the determining portion 19, first, reads out the comparison result of the elapsed time from the time comparing portion 17, and, when it is determined that the elapsed time since the cells S were seeded has passed the first replacement reference time, the instructing portion 21 issues the instruction for first medium replacement. In step S5, the determining portion 19 subsequently reads out the comparison result of the pH of the medium W from the pH comparing portion 9, and, each time it is determined that the pH of the medium W is lower than the pH replacement reference value, the instructing portion 21 issues the instruction for second and subsequent medium replacements.

In the case in which the interval replacement reference mode is selected, in step S5, the determining portion 19 reads out the comparison result of the pH of the medium W from the pH comparing portion 9 and also reads out the comparison result of the elapsed time from the time comparing portion 17, and each time it is determined that the pH of the medium W is lower than the pH replacement reference value or it is determined that the elapsed time since the cells S are seeded has passed the second replacement reference time since the medium replacement in the immediate past, the instructing portion 21 issues the instruction for the medium replacement.

In the case in which the total replacement reference mode is selected, in step S5, the determining portion 19, first, reads out the comparison result of the elapsed time from the time comparing portion 17, and, when it is determined that the elapsed time since the culturing cells S are seeded has passed the first replacement reference time, the instructing portion 21 issues the instruction for the first medium replacement. In step S5, the determining portion 19 subsequently reads out the comparison result of the pH of the medium W from the pH comparing portion 9 and also reads out the comparison result of the elapsed time from the time comparing portion 17, and, each time it is determined that the pH of the medium W is lower than the pH replacement reference value or it is determined that the elapsed time since the cells S were seeded has passed the second replacement reference time since the medium replacement in the immediate past, the instructing portion 21 issues the instruction for the second and subsequent medium replacements.

Next, an example of the cell-culturing time-series curve will be described.

In the example shown in FIG. 5, it is assumed that the total replacement reference mode is selected, the first replacement reference time is 24 hours, that is, one day, the second replacement reference time is 72 hours, that is, three days, and the pH replacement reference value is 6.9.

First, on the basis of the determination result by the determining portion 19 that the elapsed time since the seeding has passed the first replacement reference time when the elapsed time measured by the replacement-elapsed-time measuring portion 15 has passed 24 hours since the cells S were seeded, the instructing portion 21 issues the instruction for the first medium replacement, and the user manually performs the first medium replacement (first-day medium replacement).

Subsequently, on the basis of the determination result by the determining portion 19 that the elapsed time since the medium replacement in the immediate past has passed the second replacement reference time when the elapsed time measured by the replacement-elapsed-time measuring portion 15 has passed 72 hours since the first medium replacement, the instructing portion 21 issues the instruction for the second medium replacement, and the user manually performs the second medium replacement (elapsed-time medium replacement).

Subsequently, on the basis of the determination result by the determining portion 19 that the pH of the medium W is determined to be lower than pH replacement reference value when the pH of the medium W measured by the pH measuring portion 7 falls below 6.9, the instructing portion 21 issues the instruction for a third medium replacement, and the user manually performs the third medium replacement (pH medium replacement).

Subsequently, on the basis of the determination result by the determining portion 19 that the pH of the medium W is lower than the pH replacement reference value when the pH of the medium W measured by the pH measuring portion 7 falls below 6.9 again, the instructing portion 21 issues the instruction for a fourth medium replacement, and the user manually performs the fourth medium replacement (pH medium replacement).

As has been described above, with the cell culturing device 1 and the cell culturing method according to this embodiment, as a result of the user selecting one of the start replacement reference mode, the interval replacement reference mode, and the total replacement reference mode, the user is also prompted to replace the medium regardless of the pH of the medium W in the case in which the prescribed time has passed since the cells S were seeded in addition to the case in which the pH of the medium W decreases.

Accordingly, the user can execute the medium replacement without missing the timing therefor in the case in which the medium W deteriorates without the pH thereof decreasing after the seeding, in the case in which replacement with a fresh medium W is required after certain amount of time from the initiation of the culturing due to influences of a coating agent of the culturing vessel 3 and components that are included in the components of the medium W immediately after being defrosted and that are inappropriate for culturing, and so forth. Therefore, with the cell culturing device 1 and the cell culturing method according to this embodiment, it is possible to replace the medium W at an appropriate timing.

Note that examples of subject cells and recommended replacement time for each type of medium W are as follows:

With DEF-CS 500, the culturing subjects are iPS cells. and ES cells, and the recommended culturing time is 24 hours. In the case in which the cell density is low, the recommended culturing time is 24 to 48 hours.

With mTeSR1 and TeSR2, the culturing subjects are iPS cells and ES cells, and the recommended culturing time is 24 hours. In the case in which the medium quantity is doubled, the recommended culturing time is 48 hours.

With NutriStem nV9 XF, the culturing subjects are iPS cells and ES cells, and the recommended culturing time is 24 hours. In the case in which the medium quantity is doubled, the recommended culturing time is 48 hours.

With Primate ES cell medium, the culturing subjects are ES cells, and the recommended culturing time is 24 hours.

With ReproFF (Feeder-free) medium, the culturing subjects are ES cells, and the recommended culturing time is 24 hours.

With StemFit AK03N, the culturing subjects are iPS cells, and the recommended culturing time is maximally 72 hours.

### Second Embodiment

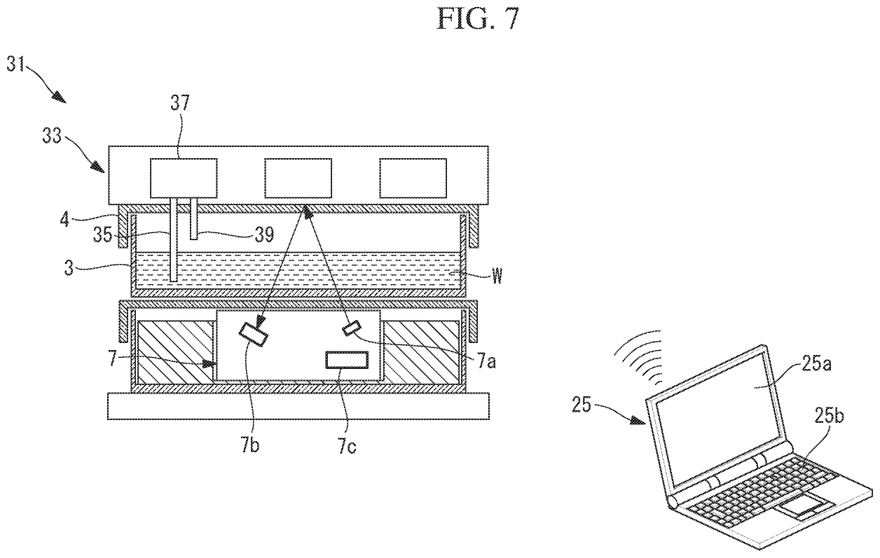

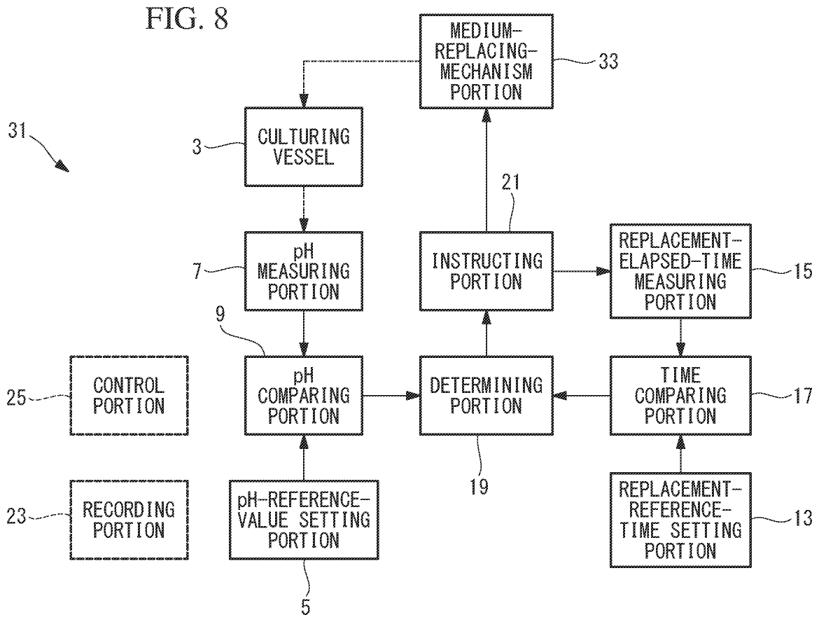

Next, a cell culturing device and a cell culturing method according to a second embodiment of the present invention will be described below with reference to FIGS. 7 and 8.

A cell culturing device 31 according to this embodiment differs from the first embodiment in that said device outputs an instruction for replacing the medium W in the culturing vessel 3 and automatically replaces the medium W on the basis of said instruction.

In describing this embodiment, the portions having the same configurations as those in the cell culturing device 1 and the cell culturing method according to the above-described first embodiment will be assigned the same reference signs, and descriptions thereof will be omitted.

The cell culturing device 31 according to this embodiment includes, for example, a medium-replacing-mechanism portion (medium replacing portion) 33 as shown in FIGS. 7 and 8.

The medium-replacing-mechanism portion 33 is a device that replaces the medium W on the basis of the instruction for medium replacement issued from the instructing portion 21 and is disposed, for example, above the culturing vessel 3, as shown in FIG. 7.

The medium-replacing-mechanism portion 33 includes: a discharge channel 35 through which the medium W in the culturing vessel 3 is sucked out; a medium retaining tank 37 that retains the old medium W that has been sucked out through the discharge channel 35 and that retains the new medium W to be supplied to the culturing vessel 3; and a supply channel 39 through which the new medium W in the medium retaining tank 37 is supplied to the culturing vessel 3.

The steps up to the instructing portion 21 outputting the instruction for the medium replacement are the same as in the first embodiment.

The control portion 25 receives the instruction for the medium replacement from the instructing portion 21, and drives the medium-replacing-mechanism portion 33 by executing the medium replacement program by means of the CPU. Then, the medium is replaced by discharging the old medium W in the culturing vessel 3 via the discharge channel 35 and by, on the other hand, supplying the new medium W in the medium retaining tank 37 via the supply channel 39.

With the cell culturing device 31 and the cell culturing method according to this embodiment, as a result of employing the medium-replacing-mechanism portion 33, it is possible to automatically replace the medium W at an appropriate timing without requiring the time and effort on the part of the user.

This embodiment can be modified as described below.

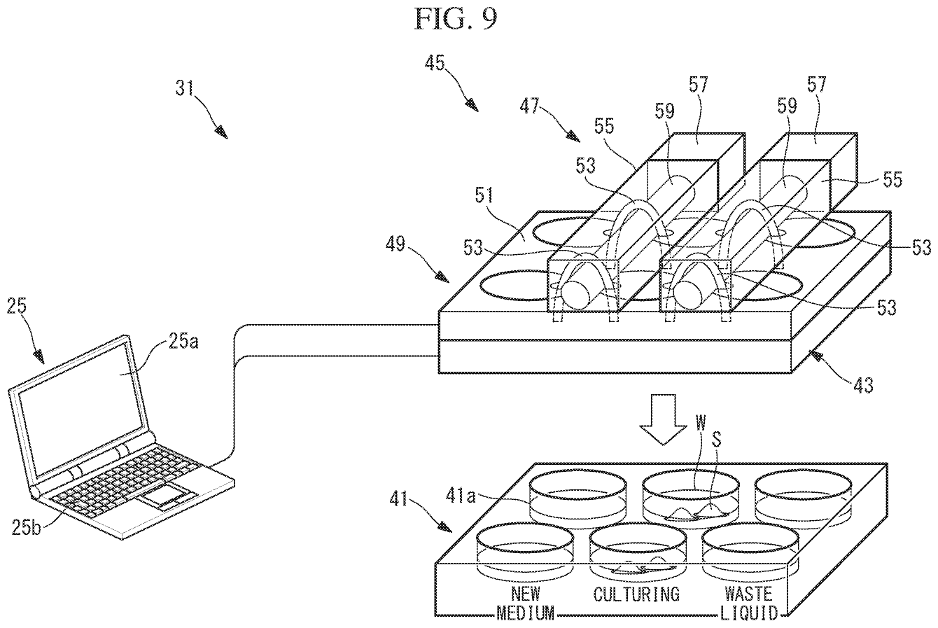

For example, as shown in FIG. 9, the cell culturing device 31 may include, instead of the pH measuring portion 7, a pH measuring portion 43 that is disposed above a multiwell plate (culturing vessel) 41 in which a plurality of wells 41a are arrayed at a certain pitch and may additionally employ, instead of the medium-replacing-mechanism portion 33, a medium-replacing-mechanism portion (medium replacing portion) 45 that is integrally formed with the pH measuring portion 43.

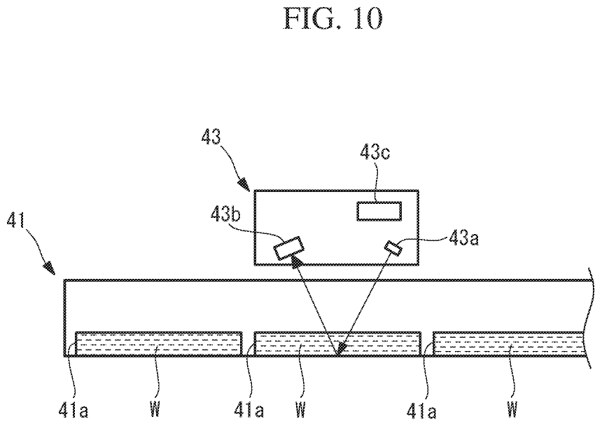

The pH measuring portion 43 is a device that optically measures the color of the medium W, as with the pH measuring portion 7, and includes, for example, as shown in FIG. 10: a light source 43a that emits light; an optical sensor 43b that detects light; and a computing portion 43c that calculates the pH of the medium W. In addition, the pH measuring portion 43 is placed on the multiwell plate 41 at the position at which the pH measuring portion 43 covers the plurality of wells 41a, as shown in FIG. 9.

As shown in FIG. 10, the pH measuring portion 43 measures the light transmittances at the medium W by means of the optical sensor 43b by irradiating the medium W, from above the wells 41a, with light beams having different wavelengths by means of the light source 43a and by detecting, by means of the optical sensor 43b, transmitted light that has been reflected upward by bottom surfaces of the wells 41a after passing through the medium W. Also, by means of the computing portion 43c, the pH of the medium W is calculated on the basis of differences among the transmittances detected by the optical sensor 43b for the respective wavelengths. The pH of the medium W calculated by the pH measuring portion 43 is transmitted to the pH comparing portion 9.

The medium-replacing-mechanism portion 45 is used in a state where the medium-replacing-mechanism portion 45 is mounted to the multiwell plate 41 and is separated therefrom by the pH measuring portion 43, as shown in FIG. 9. The medium-replacing-mechanism portion 45 includes a motive power portion 47 such as a pump and a liquid delivery portion 49.

The liquid delivery portion 49 includes: a flat-plate-like lid member 51 that is placed on the pH measuring portion 43; and a plurality of flexible tubes 53 possessing flexibility.

As a result of the respective tubes 53 passing through the lid member 51 and the pH measuring portion 43 twice in the thickness direction at positions at which the tubes 53 bridge the adjacent wells 41a when the medium-replacing-mechanism portion 45 and the pH measuring portion 43 are placed on the multiwell plate 41, both ends of each tube 53 are disposed below the lid member 51, and an intermediate position thereof, on the other hand, is disposed above the lid member 51.

In the example shown in FIG. 9, the multiwell plate 41 includes six wells 41a arranged in two rows and three columns. The respective tubes 53 are disposed so that one tube each is disposed at, in the same row, a position at which the tube bridges a well 41a in the first column and a well 41a in the second column and a position at which the tube bridges the well 41a in the second column and a well 41a in the third column. In other words, the respective tubes 53 are disposed in the lid member 51 so that two tubes are disposed in each row.

The motive power portion 47 includes: pump bodies 55; and driving portions 57 that drive the pump bodies 55. The pump bodies 55 make the medium W flow in the tubes 53 by acting on the intermediate positions in the length direction of the tubes 53 that are exposed above the lid member 51. The pump bodies 55 are, for example, peristaltic pumps or the like and deliver the medium W by being driven in a manner in which the tubes 53 are rubbed by means of rotors 59 that radially compress the tubes 53.

The driving portions 57 are, for example, motors and are turned ON/OFF, wirelessly or by means of wires, via a remote operation of the control portion 25. The user may turn ON/OFF the driving portions 57 at a desired timing by means of the control portion 25, or the control portion 25 may turn ON/OFF the driving portions 57 in accordance with a program set in advance.

The motive power portion 47 is provided so as to be attachable to/detachable from the lid member 51. It is possible to deliver the medium W in the tubes 53 by actuating the driving portions 57 in the state in which the motive power portion 47 is attached to the lid member 51. In addition, it is possible to separate the liquid delivery portion 49 and the motive power portion 47 by removing the motive power portion 47 from the lid member 51. Accordingly, for example, it is possible to configure the liquid delivery portion 49 so as to be disposable, and, on the other hand, it is possible to configure the motive power portion 47 so as to be reusable.

The instruction for the medium replacement from the instructing portion 21 is input to the medium-replacing-mechanism portion 45 via the control portion 25. When the instruction for the medium replacement is input, the medium-replacing-mechanism portion 45 collects the old medium W retained in the wells 41a by sucking out the medium W therefrom and, in replacement, supplies the new medium W to the wells 41a.

The instructing portion 21 transmits the instruction for the medium replacement to the medium-replacing-mechanism portion 45 via the control portion 25 and also transmits a reset signal for resetting the elapsed time to the replacement-elapsed-time measuring portion 15.

The replacement-elapsed-time measuring portion 15 starts to measure the elapsed time since the most recent medium replacement by using the reset signal transmitted thereto from the instructing portion 21 as a trigger.

The operation of the cell culturing device 31 and the cell culturing method according to this modification will be described below.

In order to employ the medium-replacing-mechanism portion 45, the medium W and cells S are accommodated in center wells 41a of the respective rows in the six wells 41a arranged in two rows and three columns, and the new medium W is accommodated in the wells 41a on one side of the center wells 41a, and the wells 41a on the other side of the center wells 41a are kept empty.

Next, the lid member 51 of the medium-replacing-mechanism portion 45 is disposed at the position at which the lid member 51 covers above the wells 41a accommodating the medium W and the cells S with the pH measuring portion 43 separating the lid member 51 and the wells 41a, and the end portions of the tubes 53 passing through the lid member 51 and the pH measuring portion 43 are disposed in the respective wells 41a. Accordingly, among the three wells 41a in the respective rows, the tubes 53 are individually disposed at the positions at which the tubes 53 bridge the adjacent wells 41a.

In this state, the motive power portion 47 is attached above the lid member 51. By setting the intermediate positions in the length direction of the tubes 53, which are exposed above the lid member 51, to the pump bodies 55 included in the motive power portion 47, the intermediate positions of the tubes 53 are partially compressed in the radial direction. When the driving portions 57 are actuated, the rotation of the rotors 59 moves the compressed portions of the tubes 53 in the length direction of the tubes 53, and thus, it is possible to make the medium W liquid in the interiors thereof flow in one direction.

The multiwell plate 41, on which the medium-replacing-mechanism portion 45 according to this modification is installed as described above, is accommodated in an incubator, and the cell culturing is subsequently started. The steps up to the instructing portion 21 outputting the instruction for the medium replacement are the same as in the first embodiment.

When the instruction for the medium replacement is output from the instructing portion 21, the control portion 25 causes the medium-replacing-mechanism portion 45 to be driven, and the pump body 55 installed at the tubes 53 between the center wells 41a of the respective rows and the empty wells 41a adjacent to the center wells 41a is actuated by the driving portion 57.

Accordingly, the used medium W that has been culturing the cells S in the center wells 41a is sucked into the tubes 53 by means of the motive power portion 47 and is subsequently discharged into the empty wells 41a.

Next, the pump body 55 installed at the tubes 53 between the center wells 41a and the wells 41a that are adjacent to the center wells 41a and that accommodate the new medium W is actuated by means of the driving portion 57. Accordingly, the new medium W retained in the wells 41a is sucked into the tubes 53 by means of the motive power portion 47 and is subsequently supplied to the center wells 41a.

With this modification, in the state in which the multiwell plate 41 in which the cells S are being cultured is kept accommodated in the incubator, it is possible to replace the medium W by discharging the old medium W from the wells 41a and by, on the other hand, supplying the new medium W to said wells 41a. Therefore, it is possible to reduce the time and effort related to the medium replacement on the part of the user. In addition, because the procedures do not involve placement into/removal from the incubator, it is possible to avoid stress on the cells S due to environmental changes in terms of the temperature and so forth, impacts caused when being transported, and so forth, and thus, it is possible to maintain the health of the cells S.

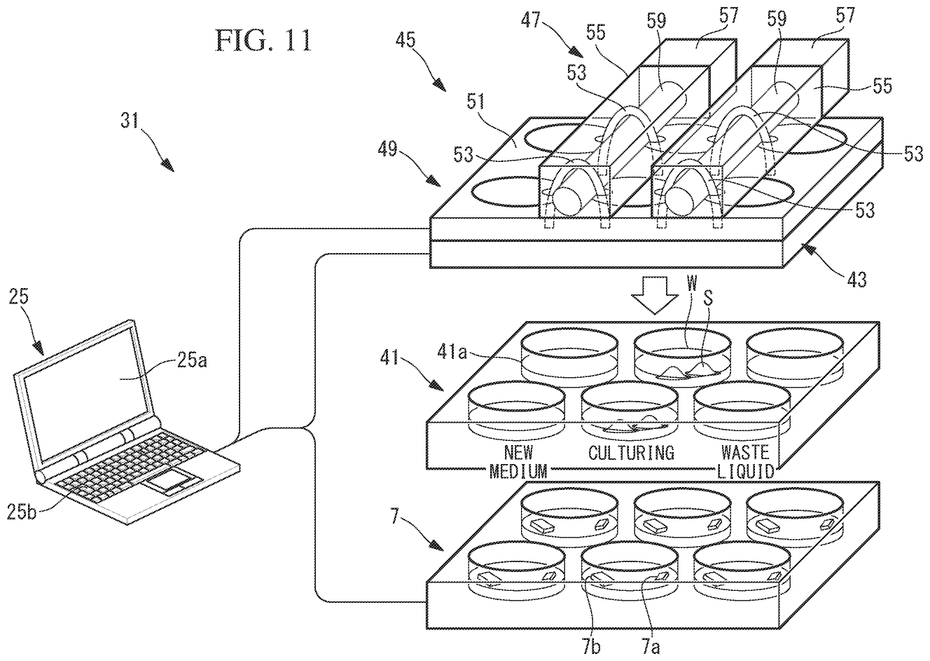

In addition, in this embodiment, for example, as shown in FIG. 11, the cell culturing device 31 may include both the pH measuring portion 7, which is installed below the multiwell plate 41, and the pH measuring portion 43, which is installed above the multiwell plate 41. Hereinafter, the pH measuring portion 7 will be assumed to be a bottom-portion pH measuring portion 7 and the pH measuring portion 43 will be assumed to be a top-portion pH measuring portion 43.

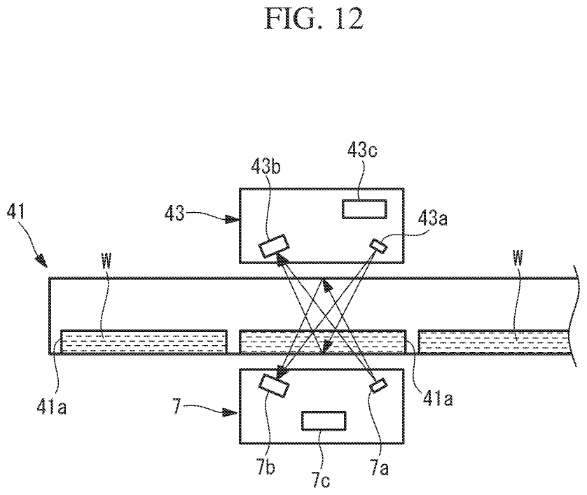

The bottom-portion pH measuring portion 7 may, for example, as shown in FIG. 12, detect, below the wells 41a by means of the optical sensor 7b, transmitted light of the light radiated onto the medium W from above the wells 41a by means of the light source 43a of the top-portion pH measuring portion 43 in addition to detecting, by means of the optical sensor 7b, transmitted light reflected downward at the top lid of the multiwell plate 41 after passing through the medium W by radiating light onto the medium W from below the wells 41a by means of the light source 7a.

As with the bottom-portion pH measuring portion 7, the top-portion pH measuring portion 43 may detect, above the wells 41a by means of the optical sensor 43b, the transmitted light of the light radiated onto the medium W from below the wells 41a by means of the light source 7a of the bottom-portion pH measuring portion 7 in addition to detecting, by means of the optical sensor 43b, transmitted light reflected upward at the bottom surfaces of the wells 41a after passing through the medium W by radiating light onto the medium W from above the wells 41a by means of the light source 43a.

In addition, the bottom-portion pH measuring portion 7 and the top-portion pH measuring portion 43 perform optical communication by utilizing the respective light sources 7a and 43a and optical sensors 7b and 43b. The top-portion pH measuring portion 43 may be integrated with the medium-replacing-mechanism portion 45 or may be a separate unit. The bottom-portion pH measuring portion 7 is controlled by the instruction from the top-portion pH measuring portion 43 and transfers the measurement operation and the measurement data to the top-portion pH measuring portion 43 in accordance with the instruction from the top-portion pH measuring portion 43.

With this modification, by disposing the bottom-portion pH measuring portion 7 and the top-portion pH measuring portion 43 so as to face each other in a manner in which the culturing vessel 3 is sandwiched therebetween, it is also possible to utilize the bottom-portion pH measuring portion 7 and the top-portion pH measuring portion 43 as transmitting-type pH measuring portions, and thus, it is possible to dispose and use said portions in accordance with the environment. Also, as a result of using the bottom-portion pH measuring portion 7 and the top-portion pH measuring portion 43 as transmitting-type pH measuring portions, it is possible to enhance the robustness as compared with the case in which the pH measuring portions are used as reflecting-type pH measuring portions.

The respective embodiments described above can be modified as below.

As a first modification, the control portion 25 may have a function for individually storing the plurality of first culturing conditions in association with a unique first replacement reference time for each of the first culturing conditions and also a function for individually storing the plurality of second culturing conditions in association with a unique second replacement reference time for each of the second culturing conditions. In addition, the replacement-reference-time setting portion 13 may read out, from the control portion 25, the first replacement reference time or the second replacement reference time corresponding to a culturing condition input by means of the input portion 25b and may set the read-out first replacement reference time or second replacement reference time in the time comparing portion 17.

In this case, when starting to culture the cells S, the user may select one of the replacement-determining operation modes and may also input the desired culturing condition by means of the input portion 25b.

Also, when the culturing condition is input by means of the input portion 25b, the replacement-reference-time setting portion 13 may read out the first replacement reference time or the second replacement reference time stored in the control portion 25 in association with the input culturing condition in accordance with the selected replacement-determining operation mode and the read-out first replacement reference time or second replacement reference time may be set to the time comparing portion 17.

For example, in the start replacement reference mode, when the user selects one of the first culturing conditions, the first replacement reference time corresponding to the selected first culturing condition may be read out from the control portion 25 by the replacement-reference-time setting portion 13. In addition, in the interval replacement reference mode, when the user selects one of the second culturing conditions, the second replacement reference time corresponding to the selected second culturing condition may be read out from the control portion 25. In addition, in the total replacement reference mode, when the user selects one of the first culturing conditions and one of the second culturing conditions, both the first replacement reference time corresponding to the selected first culturing condition and the second replacement reference time corresponding to the selected second culturing condition may be read out from the control portion 25. Then, the respective read-out replacement reference times may be set in the time comparing portion 17. Note that, in the pH replacement reference mode, no replacement reference time is read out, and the replacement reference time is not set in the time comparing portion 17.

With this modification, the user needs to simply input the desired culturing conditions, and thus, it is possible to set the first replacement reference time and the second replacement reference time in a simple manner.

In this modification, a unique replacement reference time is set for each of the culturing conditions stored in the control portion 25; however, alternatively, for example, the past culturing history may be organized into a database and the replacement reference time in accordance with the culturing situation may be set, as appropriate, on the basis of the past culturing history.

In addition, in the respective embodiments described above, the pH measuring portion 7, 43 has been described in terms of an example system in which the color of the medium W is optically measured by the light source 7a, 43a and the optical sensor 7b, 43b; however, alternatively, the pH of the medium W may be calculated on the basis of the transmittances of separate wavelengths, for example, by employing a white light source and a color sensor and by detecting, by means of the color sensor, transmitted light of light radiated onto the medium W by means of the white light source.

In addition, in the respective embodiments described above, the configurations in which the light source 7a and the optical sensor 7b are disposed in the same direction with respect to the medium W have been described as examples; however, the light source 7a and the optical sensor 7b may be disposed so as to face each other and sandwiching the medium W therebetween, and the pH may be measured on the basis of the transmittance of the transmitted light that has passed through the medium W. This is also the same for the light source 43a and the optical sensor 43b.

As above, the embodiments of the present invention have been described in detail with reference to the drawings; however, the specific configurations are not limited to these embodiments, and design alterations or the like within a range that does not depart from the scope of the present invention are also encompassed. For example, the application of the present invention is not limited to the forms in which the present invention is applied to the above-described respective embodiments; the present invention may be applied to embodiments in which these embodiments are combined, as appropriate, and is not particularly limited.

As a result, the above-described embodiments lead to the following aspect.

An aspect of the present invention is directed to a cell culturing device including: a pH measuring portion that measures a pH of a medium in which cells are being cultured; a time measuring portion that measures an elapsed time since the cells were seeded in the medium; a determining portion that determines whether or not the pH measured by the pH measuring portion is lower than a prescribed threshold and that determines whether or not the elapsed time measured by the time measuring portion has passed a prescribed time; and a replacement instructing portion that issues an instruction for medium replacement in one of the case in which the determining portion determines that the pH is lower than the prescribed threshold and the case in which the determining portion determines that the elapsed time has passed the prescribed time.

With this aspect, when the cell culturing is started by seeding the cells in the medium, the pH measuring portion measures the pH of the medium and the time measuring portion also measures elapsed time since the cells were seeded. Also, when the determining portion determines that the pH of the medium is lower than the prescribed threshold or determines that the elapsed time since the seeding has passed the prescribed time, the replacement instructing portion issues the instruction for the medium replacement.

Therefore, the user is also prompted to replace the medium regardless of the pH of the medium in the case in which the prescribed time has passed since the cells were seeded in addition to the case in which the pH of the medium decreases. Accordingly, the user can execute the medium replacement without missing the timing therefor in the case in which the medium deteriorates without the pH thereof decreasing after the seeding, in the case in which replacement to a fresh medium is required after certain amount of time from the initiation of the culturing due to influences of a coating agent of the culturing vessel and components that are included in the components of the medium immediately after being defrosted and that are inappropriate for culturing, and so forth. Therefore, with the cell culturing device according to this embodiment, it is possible to replace the medium at an appropriate timing.

In the cell culturing device according to the above-described aspect, the determining portion may determine whether or not the elapsed time has passed the first prescribed time since the cells were seeded, and the replacement instructing portion may issue, on the basis of the determination result of the determining portion that the elapsed time has passed the first prescribed time, the instruction for the first medium replacement since the cells were seeded.

With this configuration, it is possible to execute medium replacement that becomes necessary in accordance with the number of days and time after culturing is started without missing the timing therefor regardless of the pH of the medium.

In the cell culturing device according to the above-described aspect, the determining portion may determine whether or not the elapsed time has passed the second prescribed time since the medium replacement in the immediate past, and the replacement instructing portion may issue, on the basis of the determination result of the determining portion that the pH is lower than the prescribed threshold or that the elapsed time has passed the second prescribed time, the instruction for the second and subsequent medium replacement since the cells were seeded.

With this configuration, it is possible to execute, as appropriate, the second and subsequent medium replacements since the cells are seeded at the respective necessary timings in accordance with both the change in the pH of the medium and the elapsed time since the seeding.

The cell culturing device according to the above-described aspect may include: a storage portion that individually stores a plurality of first culturing conditions in association with unique first prescribed time for each of the first culturing conditions; an input portion that allows a user to input the first culturing conditions; and a setting portion that reads out, from the storage portion, the first prescribed time corresponding to the first culturing condition input by means of the input portion and that sets the read-out first prescribed time in the determining portion.

With this configuration, when the user inputs the desired first culturing conditions by means of the input portion, the setting portion sets, to the determining portion, the first prescribed time stored in the storage portion in association with the input first culturing condition. Therefore, the user needs to simply input a desired first culturing condition, and thus, it is possible to set the first prescribed time in a simple manner.

Examples of the first culturing conditions include the type of cells to be cultured, the type of vessel in which the cells are accommodated, the type of coating agent that coats the vessel, the type of culturing procedure, and so forth, as well as the type of the medium to be used.

In the cell culturing device according to the above-described aspect, the storage portion individually may store a plurality of second culturing conditions in association with a unique second prescribed time for each of the second culturing conditions; the input portion may be provided so as to also allow the user to input the second culturing conditions; and the setting portion reads out, from the storage portion, the second prescribed time corresponding to the second culturing condition input by means of the input portion and sets the read-out second prescribed time in the determining portion.

With this configuration, the user needs to simply input a desired second culturing condition, and thus, it is possible to set the second prescribed time in a simple manner by reducing the time and effort on the part of the user.

Examples of the second culturing conditions include the type of the medium to be used.

The cell culturing device according to the above-described aspect may include a notifying portion that receives the instruction for the medium replacement from the replacement instructing portion and that is capable of externally issuing a notification about the received instruction for the medium replacement.

With this configuration, the user can quickly execute the medium replacement at an appropriate timing in accordance with the instruction for the medium replacement, which is notified by the notifying portion.

The cell culturing device according to the above-described aspect may include a medium replacing portion that receives the instruction for the medium replacement from the replacement instructing portion and that replaces the medium on the basis of the received instruction for the medium replacement.

With this configuration, it is possible to automatically replace the medium at an appropriate timing by means of the medium replacing portion.

In addition, another aspect of the present invention is a cell culturing method including: seeding cells in a medium; measuring a pH of the seeded medium; measuring an elapsed time since the cells were seeded in the medium; and replacing the medium in one of the case in which it is determined that the pH is lower than the prescribed threshold and the case in which it is determined that the elapsed time has passed the prescribed time.

With this aspect, when the cell culturing is started by seeding the cells in the medium, the pH of the medium is measured and the elapsed time since the cells were seeded in the medium is also measured. Also, when it is determined that the pH of the medium is lower than the prescribed threshold or it is determined that the elapsed time since the seeding has passed the prescribed time, the medium is replaced.

Therefore, the medium is also replaced regardless of the pH of the medium in the case in which the prescribed time has passed since the cells were seeded in addition to the case in which the pH of the medium decreases. Therefore, with the cell culturing method according to this modification, it is possible to replace the medium at an appropriate timing.

In the cell culturing method according to the above-described aspect, whether or not the elapsed time has passed the first prescribed time since the cells were seeded may be determined, and a first medium replacement since the cells were seeded may be performed on the basis of a determination result that the elapsed time has passed the first prescribed time.

With this configuration, it is possible to execute medium replacement that becomes necessary in accordance with the number of days and time after culturing is started without missing the timing therefor regardless of the pH of the medium.

In the cell culturing method according to the above-described aspect, whether or not the elapsed time has passed the second prescribed time since the medium replacement in the immediate past may be determined, and second and subsequent medium replacements since the cells were seeded may be performed on the basis of the determination result that the pH is lower than the prescribed threshold or that the elapsed time has passed the second the prescribed time.

With this configuration, it is possible to execute, as appropriate, the second and subsequent medium replacements since the cells are seeded at the respective necessary timings in accordance with both the change in the pH of the medium and the elapsed time since the seeding.

In the cell culturing method according to the above-described aspect, a unique first prescribed time may be associated with each of prescribed first culturing conditions, the user may select one of the first culturing conditions, and whether or not the elapsed time has passed the first prescribed time corresponding to the first culturing condition selected by the user may be determined.

With this configuration, the elapsed time is determined by employing the first prescribed time that is unique to the desired first culturing condition selected by the user. Therefore, the user needs to simply select a desired first culturing condition, and thus, it is possible to set the first prescribed time in a simple manner by reducing the time and effort on the part of the user.

In the cell culturing method according to the above-described aspect, a unique second prescribed time may be associated with each of prescribed second culturing conditions, the user may select one of the second culturing conditions, and whether or not the elapsed time has passed the second prescribed time corresponding to the second culturing condition selected by the user may be determined.

With this configuration, the elapsed time is determined by employing the second prescribed time that is unique to the desired second culturing condition selected by the user. Therefore, the user needs to simply select a desired second culturing condition, and thus, it is possible to set the second prescribed time in a simple manner by reducing the time and effort on the part of the user.

The present invention affords an advantage in that it is possible to replace a medium at an appropriate timing.

### REFERENCE SIGNS LIST

1, 31 cell culturing device
7, 43 pH measuring portion
9 pH comparing portion (determining portion)
13 replacement-reference-time setting portion (setting portion)
15 replacement-elapsed-time measuring portion (time measuring portion)
17 time comparing portion (determining portion)
19 determining portion
21 instructing portion (replacement instructing portion)
25 control portion (storage portion, input portion, notifying portion)
33, 45 medium-replacing-mechanism portion (medium replacing portion)
W medium
S cell

1. A cell culturing device comprising:
a sensor that measures a pH of a medium in which cells are being cultured; and
one or more processors comprising hardware, the one or more processors being configured to:
measure an elapsed time since the cells were seeded in the medium;
determine whether the measured pH is lower than a prescribed threshold and whether the measured elapsed time has passed a prescribed time; and
issue an instruction for medium replacement in response to determining that the pH is lower than the prescribed threshold or in response to determining that the elapsed time has passed the prescribed time.

2. The cell culturing device according to claim 1,
wherein the determining of whether the measured pH is lower than a prescribed threshold and whether the measured elapsed time has passed a prescribed time determines whether the elapsed time has passed a first prescribed time since the cells were seeded, and
the issuing of the instruction issues, on a basis of a determination result that the elapsed time has passed the first prescribed time, the instruction for the first medium replacement since the cells were seeded.

3. The cell culturing device according to claim 2,
wherein the determining of whether the measured pH is lower than a prescribed threshold and whether the measured elapsed time has passed a prescribed time determines whether the elapsed time has passed a second prescribed time since the medium replacement in an immediate past, and
the issuing of the instruction issues, on a basis of a determination result that the pH is lower than the prescribed threshold or that the elapsed time has passed the second prescribed time, the instruction for the second and subsequent medium replacement since the cells were seeded.

4. The cell culturing device according to claim 3, further comprising:
a memory that individually stores a plurality of first culturing conditions in association with a unique time for each of the plurality of first culturing conditions,
wherein the one or more processors are further configured to:
receive a first culturing condition input by a user; and
read out, from the memory, the unique time corresponding to the received first culturing condition to set the read-out unique time as the first prescribed time.

5. The cell culturing device according to claim 4,
wherein the memory individually stores a plurality of second culturing conditions in association with a unique time for each of the plurality of second culturing conditions, and
wherein the one or more processors are further configured to:
receive a second culturing condition input by a user; and
read out, from the memory, the unique time corresponding to the received second culturing condition to set the read-out unique time as the second prescribed time.

6. The cell culturing device according to claim 1, wherein the one or more processors are further configured to receive the instruction for the medium replacement to externally issue a notification about the received instruction for the medium replacement.

7. The cell culturing device according to claim 1, wherein the one or more processors are further configured to receive the instruction for the medium replacement to replace the medium on a basis of the received instruction for the medium replacement.

8. A cell culturing method comprising:
seeding cells in a medium;
measuring a pH of the seeded medium;
measuring an elapsed time since the cells were seeded in the medium;
determining whether the measured pH is lower than a prescribed threshold and whether the measured elapsed time has passed a prescribed time; and
replacing the medium in response to determining that the pH is lower than the prescribed threshold or in response to determining that the elapsed time has passed the prescribed time.

9. The cell culturing method according to claim 8,
wherein whether the elapsed time has passed a first prescribed time since the cells were seeded is determined, and
a first medium replacement since the cells were seeded is performed on a basis of a determination result that the elapsed time has passed the first prescribed time.

10. The cell culturing method according to claim 9,
wherein whether the elapsed time has passed a second prescribed time since the medium replacement in an immediate past is determined, and
second and subsequent medium replacements since the cells were seeded are performed on a basis of a determination result that the pH is lower than the prescribed threshold or that the elapsed time has passed the second prescribed time.

11. The cell culturing method according to claim 10, further comprising:
associating a unique time with each of a plurality of first culturing conditions in advance,
selecting, by a user, a first culturing condition from the plurality of first culturing conditions, and
setting the unique time corresponding to the selected first culturing condition as the first prescribed time.

12. The cell culturing method according to claim 10, further comprising:
associating a unique time with each of a plurality of second culturing conditions in advance,
selecting, by a user, a second culturing condition from the plurality of second culturing conditions, and
setting the unique time corresponding to the selected second culturing condition as the second prescribed time.

13. A non-transitory computer readable medium storing a cell culturing program for causing a computer to execute:
receiving a pH of a medium in which cells are being cultured;
measure an elapsed time since the cells were seeded in the medium;
determining whether the received pH is lower than a prescribed threshold and whether the measured elapsed time has passed a prescribed time; and
issuing an instruction for medium replacement in response to determining that the pH is lower than the prescribed threshold or in response to determining that the elapsed time has passed the prescribed time.
